(19) United States
(12) Patent Application Publication
(10) Pub. No.: US 2016/0215266 A1
(43) Pub. Date: Jul. 28, 2016

(54) POROUS STRUCTURE FOR STEM CELL PURIFICATION AND STEM CELL CULTURE AND METHOD OF MANUFACTURING THE SAME, STEM CELL PURIFICATION DEVICE AND METHOD OF STEM CELL PURIFICATION, STEM CELL CULTIVATION DEVICE AND METHOD OF STEM CELL CULTIVATION

(71) Applicant: National Central University, TAOYUAN CITY (TW)

(72) Inventor: AKON HIGUCHI, TAOYUAN CITY (TW)

(21) Appl. No.: 14/718,074

(22) Filed: May 20, 2015

(30) Foreign Application Priority Data
Jan. 26, 2015 (TW) 104102575

(51) Int. Cl. C12N 5/0775

(52) U.S. Cl. CPC C12N 5/0667; C12N 5/0662; C12N 2533/40; C12N 2535/00

(57) ABSTRACT

A porous structure for purifying and culturing stem cells includes a substrate. The substrate has a plurality of pores. A pore diameter of the substrate ranges between 8 to 50 μm, and a material of the substrate is selected from silk, modified fiber, polyester fiber, polyurethane fiber and the combination thereof.

### RELATED APPLICATIONS

This application claims priority to Taiwanese Application Serial Number 104102575, filed Jan. 26, 2015, which is herein incorporated by reference.

### BACKGROUND

1. Field of Invention

The present invention relates to a porous structure. More particularly, the present invention relates to a porous structure for stem cell purification and method of manufacturing the same, a stem cell purification device and method of purifying stem cell and a stem cell cultivation device and method of stem cell cultivation using the same.

2. Description of Related Art

In the field of regeneration medicine, human adult stem cell, which includes human adipose derived stem cells 9hADSCs) and human bone marrow derived stem cells (hBMSCs), appeals more in comparison with human embryonic stem cells (hESCs) and human induced pluripotent stem cells (hiPSCs). The reason lies on the ethnic issues derived from human embryonic stem cells but not the human adult stem cells.

When human embryonic stem cells and human induced pluripotent stem cells are used clinically, they show strong ability in differentiation, and tumour is likely to develop from these pluripotent stem cells. In addition, the cost is relatively high in these types of cells, and the culture of human embryonic stem cells and human induced pluripotent stem cells is difficult. Compared to human adipose derived stem cells and human bone marrow stem cell, cost on the cell culture and maintenance of human embryonic stem cells and human induced pluripotent stem cells is much higher, and it is a drawback in clinical research.

Although human adipose derived stem cells and human bone marrow stem cells are likely to be used in regeneration medicine, especially in cell therapy and tissue engineering, in comparison with human embryonic stem cells and human induced pluripotent stem cells, human adipose derived stem cells and human bone marrow stem cells lack the ability of differentiation and pluripotency. The deficiency results from heterogeneity of human adipose derived stem cells and human bone marrow stem cells. Human adipose derived stem cells and human bone marrow stem cells are non-homogenous cells, but they can perform different differentiation to become multiple specific branch of cells.

In general, purifying pluripotent and pure human adipose derived stem cells from adipose tissue in a cell purification device can improve the efficiency of cell therapy and tissue engineering where human adipose derived stem cells are used. Therefore, there is an urgent need calling for a porous structure and a method of making the same for better purification and cell culture.

### SUMMARY

According to the issue addressed previously, the instant disclosure provides a novel porous structure for purification and stem cell culture and method of making the same. The stem cell purification device and stem cell cultivation device help to produce human adipose derived stem cells and human bone marrow stem cells with relatively higher pluripotency and purity in a more efficient approach. The overall cost of stem cell purification and stem cell culture is greatly reduced.

According to an embodiment of the instant disclosure, a porous structure for purifying and culturing stem cells is provided. The porous structure includes a substrate. The substrate has a plurality of pores. A pore diameter of the substrate ranges between 8 to 50 μm, and a material of the substrate is selected from silk, modified fiber, polyester fiber, polyurethane fiber and the combination thereof.

According to an embodiment of the instant disclosure, when the material is polyurethane fiber, the pore diameter is approximately 11 μm, and the polyurethane fiber contains polyurethane.

According to an embodiment of the instant disclosure, when the material is modified fiber, the pore diameter is approximately 8 μm, and the modified fiber contains nitrocellulose.

According to an embodiment of the instant disclosure, when the material is the polyester fiber, the pore diameter is approximately 11 μm, and the polyester fiber contains nylon.

According to an embodiment of the instant disclosure, when the material is the silk, the substrate further includes a poly(lactide-co-glycolic acid) (PLGA) layer disposed on a surface of the silk substrate, and the PLGA/silk porous structure made of the PLGA layer and the silk has the pore diameter of approximately 11 μm. The PLGA layer is made of a polymer, and the polymer has a formula as shown below where x and y are independent integral such that the polymer has a molecular weight between 60,000 to 110,000.

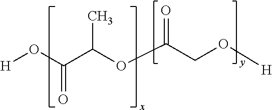

According to an embodiment of the instant disclosure, the silk includes silkworm silk, spider silk and the combination thereof.

The instant disclosure also provides a method of manufacturing a porous structure for separating stem cells. The method includes providing a substrate, and the substrate is formed with a plurality of pores. A pore diameter of the substrate ranges between 8 to 50 μm, and a material of the substrate is selected from silk, modified fiber, polyester fiber, polyurethane fiber and the combination thereof.

According to an embodiment of the instant disclosure, when the material is the polyurethane fiber, the pore diameter is approximately 11 μm, and the polyurethane fiber contains polyurethane.

According to an embodiment of the instant disclosure, when the material is the modified fiber, the pore diameter is approximately 8 μm, and the modified fiber includes nitrocellulose.

According to an embodiment of the instant disclosure, when the material is polyester fiber, the pore diameter is approximately 11 μm, and the polyester fiber includes nylon.

According to an embodiment of the instant disclosure, when the material is silk, the method further includes soaking the substrate in a poly(lactide-co-glycolic acid) (PLGA) solution such that the PLGA solution forms a PLGA layer on a surface of the substrate. The PLGA/silk porous structure made of the PLGA layer and the silk has the pore diameter of approximately 11 μm. The PLGA solution is made of PLGA and a first organic solvent, and the PLGA has a formula shown below where x and y are independent integral such that the polymer has a molecular weight between 60,000 to 110,000.

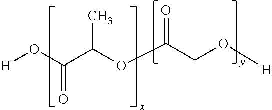

According to an embodiment of the instant disclosure, the silk includes silkworm silk, spider silk and the combination thereof.

According to an embodiment of the instant disclosure, the first organic solvent is dimethyl sulfoxide (DMSO).

According to an embodiment of the instant disclosure, the PLGA solution has a PLGA concentration ranging between 3-15 wt %.

According to an embodiment of the instant disclosure, the method further includes soaking the substrate in the PLGA solution at −20° C. for 24 hours.

According to an embodiment of the instant disclosure, the method further includes soaking the PLGA/silk porous structure made of the PLGA layer and the silk in a second organic solvent at −20° C. for three days. The second organic solvent is changed twice a day to obtain an intermediate.

According to an embodiment of the instant disclosure, the second organic solvent is 75% (v/v) ethanol solution.

According to an embodiment of the instant disclosure, the method further includes drying the intermediate at a ventilating condition for three days and then resting the intermediate at a vacuum condition to dry for 24 hours to obtain the porous structure.

The instant disclosure also provides a stem cell purification device. The device includes a first substrate, a second substrate and the porous structure as previously described. The first substrate is formed with a first opening. The second substrate is formed with a second opening. The porous structure is sandwiched between the first and second substrates.

The instant disclosure also provides a method of purifying stem cell. The method includes providing a primary cell mixture and adding the primary cell mixture to the first opening of the first substrate of the stem cell purifying device and receiving an eluate from the second opening of the second substrate. Subsequently, the stem cell purifying device is inversed, a first washing liquid is added to the second opening of the second substrate, and finally a recovered solution from the first opening of the first substrate is received.

According to an embodiment of the instant disclosure, in the step of providing the primary cell mixture further includes decomposing an adipose tissue by a collagenase. The primary cell mixture contains human adipose derived stem cells (hADSCs).

According to an embodiment of the instant disclosure, the eluate contains human adipose derived stem cells (hADSCs).

According to an embodiment of the instant disclosure, the recovered solution contains human adipose derived stem cells (hADSCs).

According to an embodiment of the instant disclosure, the washing liquid is a cell culture medium.

The instant disclosure also provides a stem cell cultivation device. The stem cell cultivation device includes a container, cell culture medium and the porous structure as previously described. The container defines a receiving space. The cell culture medium is received by the receiving space of the container.

According to an embodiment of the instant disclosure, the container is a culture dish.

The instant disclosure also provides a method of culturing stem cell. The method includes purifying a primary cell mixture by the porous structure as previously described. Then, the porous structure is soaked in the cell culture medium. A surface of the porous structure has a plurality of stem cells.

According to an embodiment of the instant disclosure, the stem cells include human adipose derived stem cells.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a top view showing a porous structure for purifying and culturing stem cells in accordance with an embodiment of the instant disclosure;

FIGS. 2A to 2B are schematic diagram showing a method of purifying and culturing stem cells in accordance with an embodiment of the instant disclosure;

FIG. 3 is an exploded view showing a device for purifying stem cell in accordance with an embodiment of the instant disclosure;

FIG. 4 is a schematic diagram showing steps of a method of purifying stem cells in accordance with an embodiment of the instant disclosure;

FIG. 5 is an exploded view showing a device for culturing stem cells in accordance with an embodiment of the instant disclosure; and

FIG. 6 is a schematic view showing steps of a method of culturing stem cells in accordance with an embodiment of the instant disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

To increase the efficiency of conventional human adipose derived stem cells purification and culture and reduce production cost thereof, the instant disclosure provides a porous structure for purifying and culturing stem cells and method of manufacturing the same. The resulting stem cell purification device and culture device improve the purification efficiency of human adipose derived stem cells, and the production cost goes down altogether.

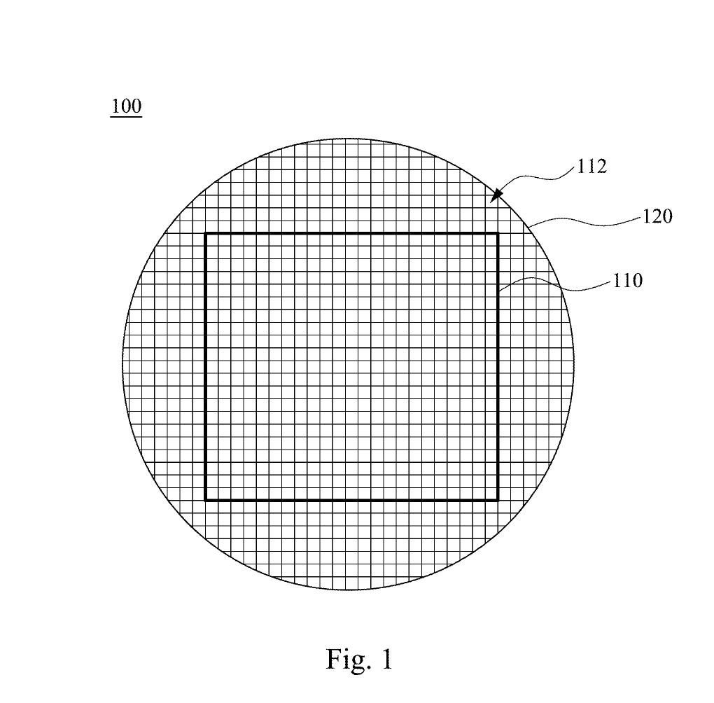

FIG. 1 is a top view showing a porous structure for purifying and culturing stem cells. As shown in FIG. 1, a porous structure 100 for purifying and culturing stem cells includes a substrate 110. The substrate 110 is formed with a plurality of pores 112. A pore diameter of the pore 112 ranges between 8 and 50 μm. The material of the substrate 110 is selected from silk, modified fiber, polyester, polyurethane (PU) and the combination thereof.

According to an embodiment of the instant disclosure, when the material of the substrate 110 is the polyurethane fiber, the pore 112 has the pore diameter of approximately 11 μm. The polyurethane fiber contains polyurethane. Given that an expected result of stem cell purification and culture is achieved, the pore diameter of the pore 112 of the substrate 110, which is made of polyurethane, may range from 8 to 20 μm. When the pore diameter of the pore 112 is approximately 11 μm, the results of stem cell purification and culture are at their best.

According to an embodiment of the instant disclosure, when the material of the substrate 110 is the modified fiber, a pore diameter of the pore 112 is approximately 8 μm. The modified fiber contains nitrocellulose. Given that an expected result of stem cell purification and culture is achieved, the pore diameter of the pore 112 of the substrate 110, which is made of nitrocellulose, may range from 5 to 15 μm. When the pore diameter of the pore 112 is approximately 8 μm, the results of stem cell purification and culture are at their best.

According to an embodiment of the instant disclosure, when the material of the substrate 110 is the polyester fiber, a pore diameter of the pore 112 is approximately 11 μm. The polyester fiber contains nylon. Given that an expected result of stem cell purification and culture is achieved, the pore diameter of the pore 112 of the substrate 110, which is made of nylon, may range from 8 to 20 μm. When the pore diameter of the pore 112 is approximately 11 μm, the results of stem cell purification and culture are at their best.

According to an embodiment of the instant disclosure, when the material of the substrate 110 is the silk, a pore diameter of the pore 112 is approximately 11 μm. The silk contains a poly(lactide-co-glycolic acid) (PLGA) layer 120 disposed on a surface of the substrate 110 made of the silk. The pore diameter of the PLGA/silk porous structure 100, which is made of the substrate from PLGA layer 120 and silk, is approximately 11 μm. Given that an expected result of stem cell purification and culture is achieved, the pore diameter of the pore 112 of the substrate 110, which is made of PLGA layer 120 and silk, may range from 8 to 20 μm. When the pore diameter of the pore 112 is approximately 11 μm, the results of stem cell purification and culture are at their best.

The PLGA layer 120 is composed of a polymer, the polymer has a structure shown in formula (I) where x and y are independent integrals such that the polymer has a molecular weight ranging between 60,000 and 110,000.

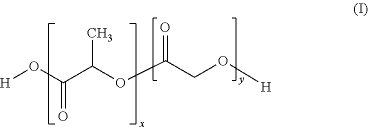

According to an embodiment of the instant disclosure, the silk includes silkworm silk, spider silk and the combination thereof.

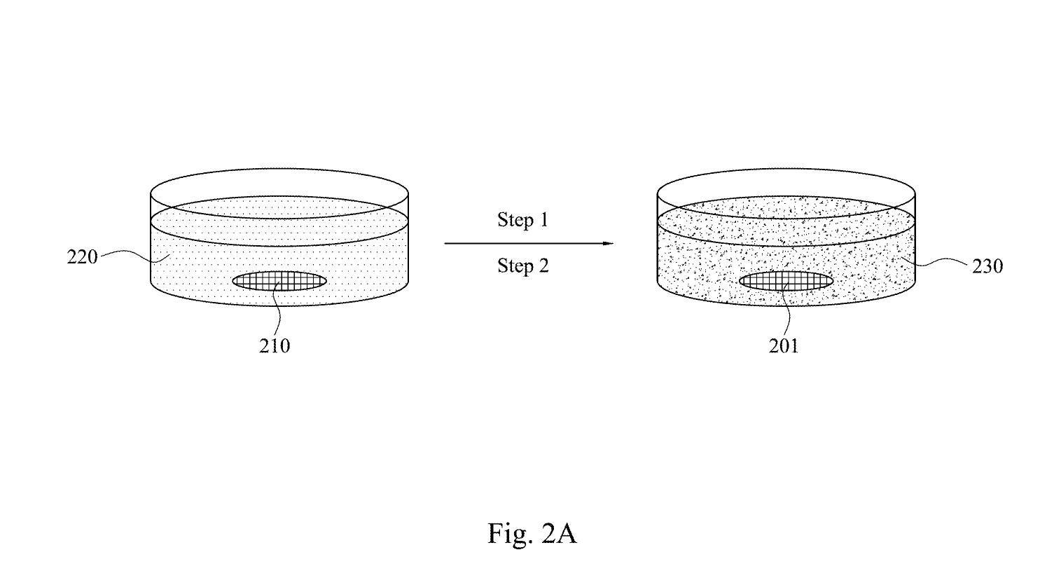

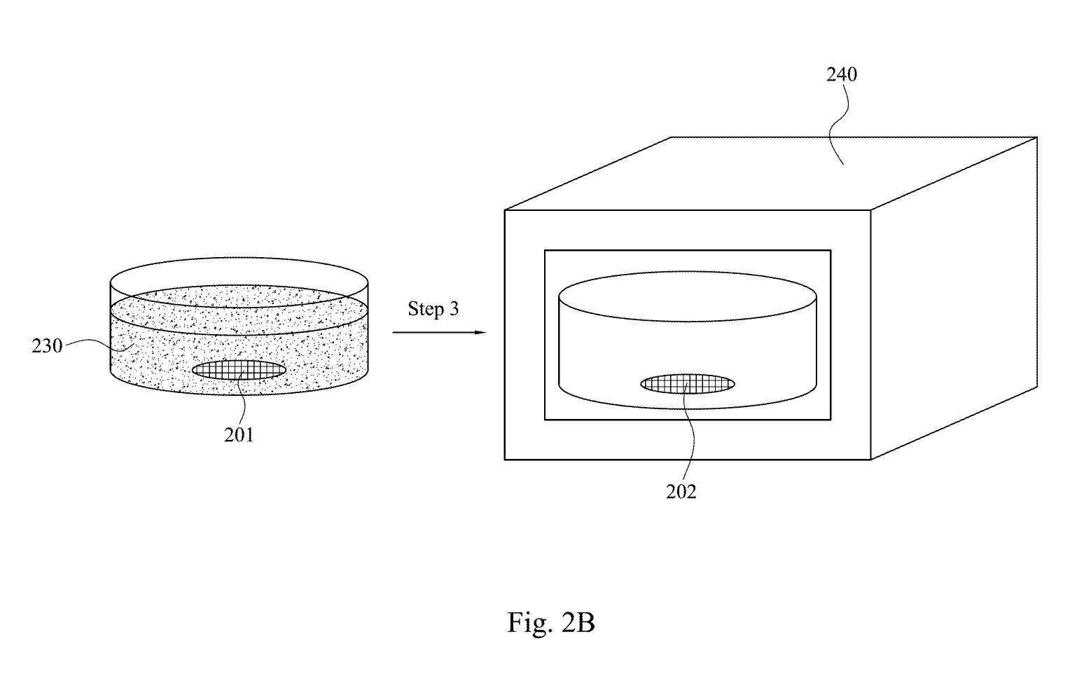

FIGS. 2A to 2B are schematic diagram showing a method of purifying and culturing stem cells in accordance with an embodiment of the instant disclosure. The method includes formation of a first structure and removal of a first organic solvent in the first structure to dry, and finally a porous structure is obtained.

The method includes providing a substrate. A pore diameter of the substrate ranges between 8 to 50 μm, and a material of the substrate is selected from silk, modified fiber, polyester fiber, polyurethane fiber and the combination thereof.

According to an embodiment of the instant disclosure, when the material of the substrate is the polyurethane fiber, the pore has the pore diameter of approximately 11 μm. The polyurethane fiber contains polyurethane. Given that an expected result of stem cell purification and culture is achieved, the pore diameter of the pore of the substrate, which is made of polyurethane, may range from 8 to 20 μm. When the pore diameter of the pore 112 is approximately 11 μm, the results of stem cell purification and culture are at their best.

According to an embodiment of the instant disclosure, when the material of the substrate is the modified fiber, a pore diameter of the pore is approximately 8 μm. The modified fiber contains nitrocellulose. Given that an expected result of stem cell purification and culture is achieved, the pore diameter of the pore of the substrate, which is made of nitrocellulose, may range from 5 to 15 μm. When the pore diameter of the pore 112 is approximately 8 μm, the results of stem cell purification and culture are at their best.

According to an embodiment of the instant disclosure, when the material of the substrate is the polyester fiber, a pore diameter of the pore is approximately 11 μm. The polyester fiber contains nylon. Given that an expected result of stem cell purification and culture is achieved, the pore diameter of the pore of the substrate, which is made of nylon, may range from 8 to 20 μm. When the pore diameter of the pore is approximately 11 μm, the results of stem cell purification and culture are at their best.

As shown in FIG. 2A, the first structure 201 is formed. When the material of the substrate 210 is the silk, the substrate 210 is soaked in a poly(lactide-co-glycolic acid) (PLGA) solution 220 such that the PLGA solution 220 forms a PLGA layer (not shown) on a surface of the substrate 210. The substrate 210 is formed with a plurality of pores, and a pore diameter of the first structure 201, which is made of the PLGA layer and silk, is approximately 11 μm. Given that an expected result of stem cell purification and culture is achieved, the pore diameter of the pore of the substrate, which is made of PLGA layer and silk, may range from 8 to 20 μm. When the pore diameter of the pore is approximately 11 μm, the results of stem cell purification and culture are at their best.

The PLGA solution 220 is composed of PLGA and a first organic solvent, PLGA has a structure shown in formula (I) where x and y are independent integrals such that the polymer has a molecular weight ranging between 60,000 and 110,000.

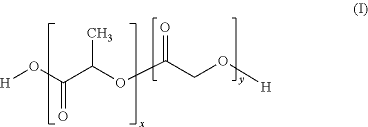

According to an embodiment of the instant disclosure, the silk includes silkworm silk, spider silk and the combination thereof. According to an embodiment of the instant disclosure, the first organic solvent is dimethyl sulfoxide (DMSO). According to an embodiment of the instant disclosure, the polymer solution is a PLGA solution, and the PLGA solution has a PLGA concentration ranging between 3-15 wt %. According to an embodiment of the instant disclosure, the polymer solution is a DMSO solution containing PLGA, and the concentration of PLGA may be 3, 5, 10 or 15 wt %.

As shown in FIG. 2A, in step 1 of forming the first structure 201 includes soaking the substrate 210 in the PLGA solution 220 at −20° C. for 24 hours. Subsequently, in step 2 of removing the first organic solvent in the first structure 201 includes soaking the first structure 201 in a second organic solvent 230 at −20° C. for 3 days. The second organic solvent 230 is changed twice a day to obtain an intermediate (i.e., a second structure 202). According to an embodiment of the instant disclosure, the second organic solvent is 75% (v/v) ethanol solution.

As shown in FIG. 2B, step 3 includes resting the second structure 202 in a ventilating condition (not shown) for 3 days. Then, the second structure 202 rests in a vacuum condition 240 for drying for 24 hours so as to obtain the PLGA/silk porous structure 100 as shown in FIG. 1.

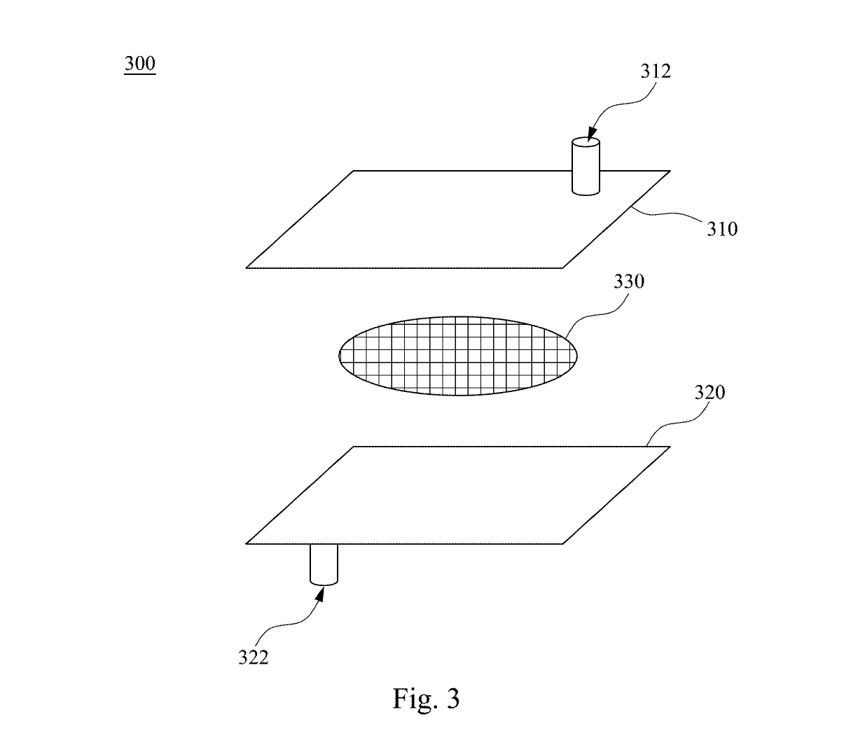

FIG. 3 is an exploded view showing a device for purifying stem cell in accordance with an embodiment of the instant disclosure. As shown in FIG. 3, the stem cell purification device 300 includes a first substrate 310, a second substrate 320 and a porous structure 330. The first substrate 310 is formed with a first opening 312. The second substrate 320 is formed with a second opening 322. The porous structure 330 is sandwiched between the first and second substrates 310, 330.

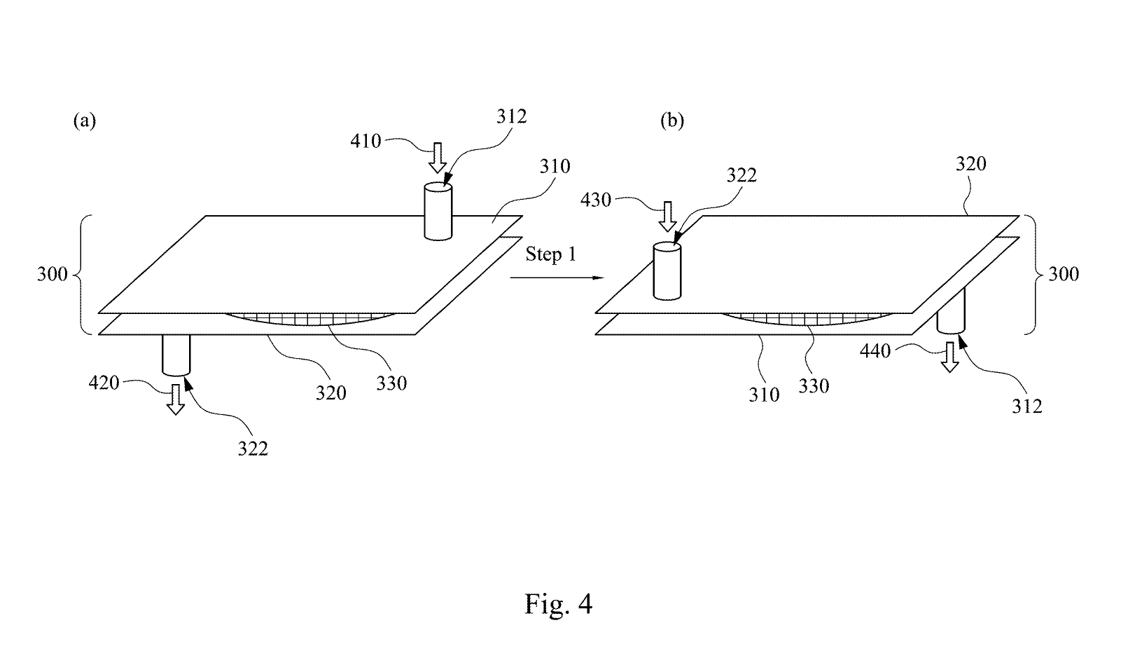

FIG. 4 is a schematic diagram showing steps of a method of purifying stem cells in accordance with an embodiment of the instant disclosure. The method of purifying stem cells includes the following steps. A primary cell mixture is provided. The primary cell mixture is added to the first opening of the first substrate in the previously described stem cell purification device. An eluate flows out from the second opening of the second substrate. The stem cell purification device is then inversed to an up side down position. A washing liquid is added to the second substrate via the second opening. Subsequently, a recovered solution is obtained from the first opening of the first substrate. According to an embodiment of the instant disclosure, n the step of providing the primary cell mixture further includes decomposing an adipose tissue by a collagenase. The primary cell mixture contains human adipose derived stem cells (hADSCs).

As shown in FIG. 4(a), the primary cell mixture 410 is added to the first opening 312 of the substrate 310 of the stem cell purification device 300. After the filtering and purification process via the porous structure 330, the eluate 420 flows out of the second substrate 320 via the second opening 322. According to an embodiment of the instant disclosure, the eluate 420 contains human adipose derived stem cells (hADSCs).

In the step 1 shown in FIG. 4, the stem cell purification device 300 is inversed in an up side down position. That is to say, the second substrate 320 is on top of the first substrate 310, and the first opening 312 of the first substrate 310 is directed downwardly, while the second opening 322 of the second substrate 320 is directed upwardly.

As shown in FIG. 4(b), the washing liquid 430 is added to the second substrate 320 through the second opening 322, and the recovered solution 440 is received from the first opening 312 of the first substrate 310. According to an embodiment of the instant disclosure, the washing liquid 430 is a cell culture medium. According to an embodiment of the instant disclosure, the recovered solution 440 contains human adipose derived stem cells (hADSCs).

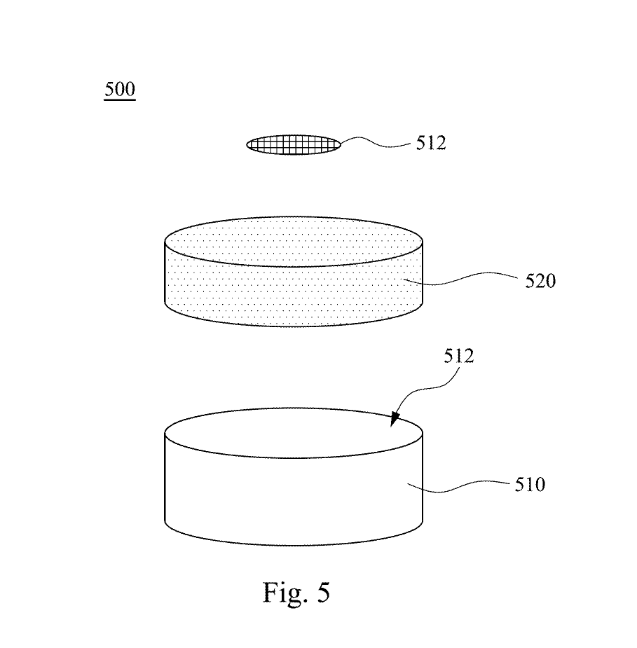

FIG. 5 is an exploded view showing a device for culturing stem cells in accordance with an embodiment of the instant disclosure. As shown in FIG. 5, the stem cell cultivation device 500 includes a container 510, cell culture medium 520 and a porous structure 530. The container 510 defines a receiving space 512. According to an embodiment of the instant disclosure, the container 510 is a culture dish. The cell culture medium 520 is received by the receiving space 512 of the container 510. The porous structure 530 is soaked in the cell culture medium 520. A surface of the porous structure 530 has a plurality of stem cells (not shown).

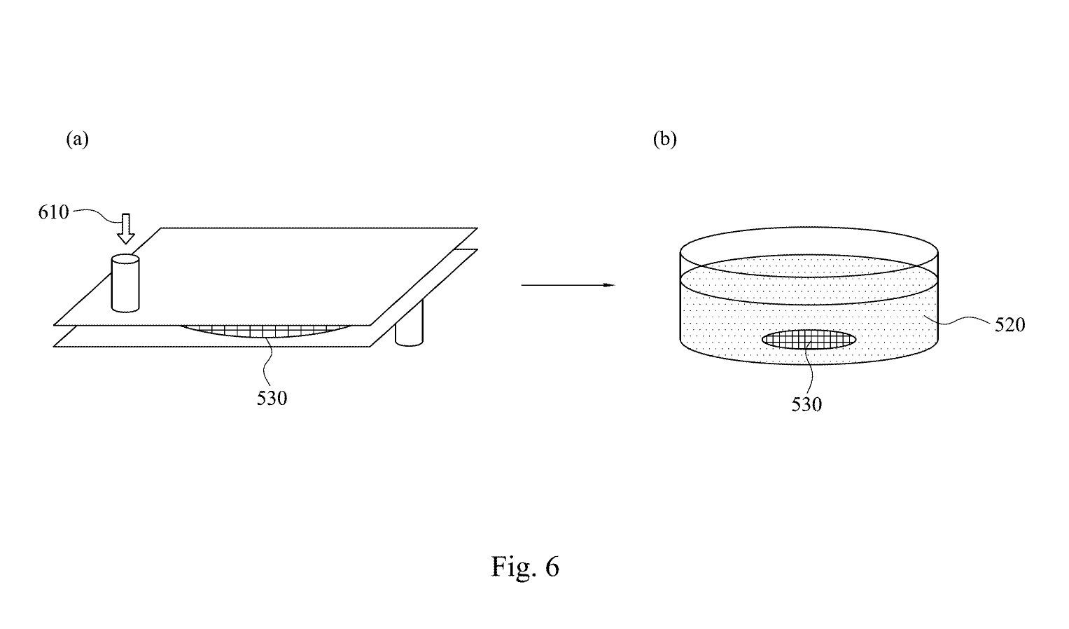

FIG. 6 is a schematic view showing steps of a method of culturing stem cells in accordance with an embodiment of the instant disclosure. The method of stem cell cultivation includes the following steps. A primary cell mixture is provided by the previously described filtration and purification using the porous structure. The porous structure is soaked in the cell medium. A surface of the porous structure has a plurality of stem cells.

As shown in FIG. 6(a), the primary cell mixture 610 is filtered and purified by the porous structure 530. Subsequently, as shown in FIG. 6(b), the porous structure 530 is soaked in cell culture medium 520. A surface of the porous structure 530 has a plurality of stem cells (not shown). According to an embodiment of the instant disclosure, the stem cells include human adipose derived stem cells.

According to some embodiments of the instant disclosure, the porous structure for purifying and culturing stem cells exhibits higher efficiency in purifying pluripotent and pure human adipose derived stem cell so as to reduce overall stem cell purification and cultivation production cost. According to some embodiments of the instant disclosure, the stem cell cultivation device arranges the porous structure in between two substrates. By flipping the device up side down, target stem cells can be separated and purified. According to some embodiments of the instant disclosure, after the target stem cells are separated and purified, the target stem cell will be attached to the surface of the porous structure. At the same time, the porous structure having target stem cells attached on its surface is soaked in the cell culture medium to continue culturing the target stem cell. The growth rate and pluripotency of the target stem cell can therefore be promoted.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A porous structure for purifying and cultivating stem cells, comprising:
a substrate having a plurality of pores,
wherein the pores in the substrate has a pore diameter ranging between 8 to 50 μm and a material of the substrate is selected from silk, modified fiber, polyester fiber, polyurethane fiber and the combination thereof,
when the material is the polyurethane fiber, the pore diameter is approximately 11 μm, and the polyurethane fiber contains polyurethane,
when the material is the modified fiber, the pore diameter is approximately 8 μm, and the modified fiber contains nitrocellulose,
when the material is the polyester fiber, the pore diameter is approximately 11 μm, and the polyester fiber contains nylon,
when the material is the silk, the substrate further comprises a poly(lactide-co-glycolic acid) (PLGA) layer disposed on a surface of the silk substrate, and the PLGA/silk porous structure made of the PLGA layer and the silk has the pore diameter of approximately 11 μm, wherein the PLGA layer is made of a polymer, the polymer has a formula: 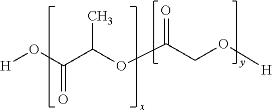
wherein x and y are independent integral such that the polymer has a molecular weight between 60,000 to 110,000.

2. The porous structure of claim 1, wherein the silk includes silkworm silk, spider silk and the combination thereof.

3. A method of manufacturing porous structure for separating stem cells comprising:
providing a substrate, the substrate formed with a plurality of pores, wherein a pore diameter ranges between 8 to 50 μm, and a material of the substrate is selected from silk, modified fiber, polyester fiber, polyurethane fiber and the combination thereof,
when the material is the polyurethane fiber, the pore diameter is approximately 11 μm, and the polyurethane fiber contains polyurethane,
when the material is the modified fiber, the pore diameter is approximately 8 μm, the modified fiber includes nitrocellulose,
when the material is polyester fiber, the pore diameter is approximately 11 μm, and the polyester fiber includes nylon,
when the material is silk, the method further comprises soaking the substrate in a poly(lactide-co-glycolic acid) (PLGA) solution such that the PLGA solution forms a PLGA layer on a surface of the substrate, wherein the PLGA/silk porous structure made of the PLGA layer and the silk has the pore diameter of approximately 11 μm, wherein the PLGA solution is made of PLGA and a first organic solvent, the PLGA has a formula: 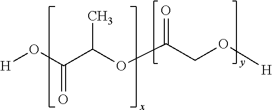
wherein x and y are independent integral such that the polymer has a molecular weight between 60,000 to 110,000; and
removing the first organic solvent to dry.

4. The method of claim 3, wherein the silk includes silkworm silk, spider silk and the combination thereof.

5. The method of claim 3, wherein the first organic solvent is dimethyl sulfoxide (DMSO).

6. The method of claim 3, wherein the PLGA solution has a PLGA concentration ranging between 3-15 wt %.

7. The method of claim 3 further comprising soaking the substrate in the PLGA solution at −20° C. for 24 hours.

8. The method of claim 3 further comprising soaking the PLGA/silk porous structure made of the PLGA layer and the silk in a second organic solvent at −20° C. for three days, wherein the second organic solvent is changed twice a day to obtain an intermediate.

9. The method of claim 8, wherein the second organic solvent is 75% (v/v) ethanol solution.

10. The method of claim 8 further comprising:
drying the intermediate at a ventilating condition for three days; and
resting the intermediate at a vacuum condition to dry for 24 hours to obtain the porous structure.

11. A stem cell purifying device comprising:
a first substrate formed with a first opening;
a second substrate formed with a second opening; and
a porous structure sandwiched between the first and second substrates, wherein the porous structure comprises:
a substrate having a plurality of pores,
wherein the pores in the substrate has a pore diameter ranging between 8 to 50 μm and a material of the substrate is selected from silk, modified fiber, polyester fiber, polyurethane fiber and the combination thereof,
when the material is the polyurethane fiber, the pore diameter is approximately 11 μm, and the polyurethane fiber contains polyurethane,
when the material is the modified fiber, the pore diameter is approximately 8 μm, and the modified fiber contains nitrocellulose,
when the material is the polyester fiber, the pore diameter is approximately 11 μm, and the polyester fiber contains nylon,
when the material is the silk, the substrate further comprises a poly(lactide-co-glycolic acid) (PLGA) layer disposed on a surface of the silk substrate, and the PLGA/silk porous structure made of the PLGA layer and the silk has the pore diameter of approximately 11 μm, wherein the PLGA layer is made of a polymer, the polymer has a formula: 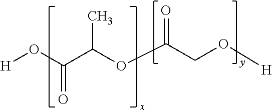
wherein x and y are independent integral such that the polymer has a molecular weight between 60,000 to 110,000.

12. A method of purifying stem cells comprising:
providing a primary cell mixture;
adding the primary cell mixture to the first opening of the first substrate of a stem cell purifying device and receiving an eluate from the second opening of the second substrate; and
inversing the stem cell purifying device, adding a first washing liquid to the second opening of the second substrate, and receiving a recovered solution from the first opening of the first substrate;
wherein the stem cell purifying device comprising:
a first substrate formed with a first opening;
a second substrate formed with a second opening; and
a porous structure sandwiched between the first and second substrates, wherein the porous structure comprises:
a substrate having a plurality of pores,
wherein the pores in the substrate has a pore diameter ranging between 8 to 50 μm and a material of the substrate is selected from silk, modified fiber, polyester fiber, polyurethane fiber and the combination thereof,
when the material is the polyurethane fiber, the pore diameter is approximately 11 μm, and the polyurethane fiber contains polyurethane,
when the material is the modified fiber, the pore diameter is approximately 8 μm, and the modified fiber contains nitrocellulose,
when the material is the polyester fiber, the pore diameter is approximately 11 μm, and the polyester fiber contains nylon,
when the material is the silk, the substrate further comprises a poly(lactide-co-glycolic acid) (PLGA) layer disposed on a surface of the silk substrate, and the PLGA/silk porous structure made of the PLGA layer and the silk has the pore diameter of approximately 11 μm, wherein the PLGA layer is made of a polymer, the polymer has a formula: 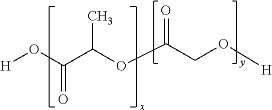
wherein x and y are independent integral such that the polymer has a molecular weight between 60,000 to 110,000.

13. The method of purifying stem cells of claim 12, wherein in the step of providing the primary cell mixture further comprises decomposing a adipose tissue by a collagenase, wherein the primary cell mixture contains human adipose derived stem cells (hADSCs).

14. The method of purifying stem cells of claim 12, wherein the eluate contains human adipose derived stem cells (hADSCs).

15. The method of purifying stem cells of claim 12, wherein the recovered solution contains human adipose derived stem cells (hADSCs).

16. The method of purifying stem cells of claim 12, wherein the washing liquid is a cell culture medium.

17. A stem cell cultivation device comprising:
a container defining a receiving space;
a cell culture medium received by the receiving space of the container; and
a porous structure disposed in the cell culture medium, wherein a surface of the porous structure includes a plurality of stem cells,
wherein the porous structure comprises:
a substrate having a plurality of pores,
wherein the pores in the substrate has a pore diameter ranging between 8 to 50 μm and a material of the substrate is selected from silk, modified fiber, polyester fiber, polyurethane fiber and the combination thereof,
when the material is the polyurethane fiber, the pore diameter is approximately 11 μm, and the polyurethane fiber contains polyurethane,
when the material is the modified fiber, the pore diameter is approximately 8 μm, and the modified fiber contains nitrocellulose,
when the material is the polyester fiber, the pore diameter is approximately 11 μm, and the polyester fiber contains nylon,
when the material is the silk, the substrate further comprises a poly(lactide-co-glycolic acid) (PLGA) layer disposed on a surface of the silk substrate, and the PLGA/silk porous structure made of the PLGA layer and the silk has the pore diameter of approximately 11 μm, wherein the PLGA layer is made of a polymer, the polymer has a formula: 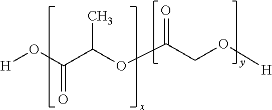
wherein x and y are independent integral such that the polymer has a molecular weight between 60,000 to 110,000.

18. The stem cell cultivation device of claim 17, wherein the container is a culture dish.

19. A method of culturing stem cell comprising:
purifying a primary cell mixture by a porous structure; and
soaking the porous structure in a cell culture medium, wherein a surface of the porous structure has a plurality of stem cells;
wherein the porous structure comprises:
a substrate having a plurality of pores,
wherein the pores in the substrate has a pore diameter ranging between 8 to 50 μm and a material of the substrate is selected from silk, modified fiber, polyester fiber, polyurethane fiber and the combination thereof,
when the material is the polyurethane fiber, the pore diameter is approximately 11 μm, and the polyurethane fiber contains polyurethane,
when the material is the modified fiber, the pore diameter is approximately 8 μm, and the modified fiber contains nitrocellulose,
when the material is the polyester fiber, the pore diameter is approximately 11 μm, and the polyester fiber contains nylon,
when the material is the silk, the substrate further comprises a poly(lactide-co-glycolic acid) (PLGA) layer disposed on a surface of the silk substrate, and the PLGA/silk porous structure made of the PLGA layer and the silk has the pore diameter of approximately 11 μm, wherein the PLGA layer is made of a polymer, the polymer has a formula: 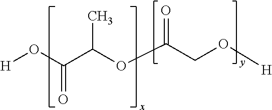
wherein x and y are independent integral such that the polymer has a molecular weight between 60,000 to 110,000.

20. The method of culturing stem cells of claim 19, wherein the stem cells include human adipose derived stem cells.
